(19) United States
(12) Patent Application Publication
(10) Pub. No.: US 2008/0208482 A1
(43) Pub. Date: Aug. 28, 2008

(54) Display Producing System

(76) Inventor: John Charles Augustus David Buchan, Norfolk (GB)

Correspondence Address: WORKMAN NYDEGGER, 60 EAST SOUTH TEMPLE, 1000 EAGLE GATE TOWER, SALT LAKE CITY, UT, 84111, US

(21) Appl. No.: 11/816,046

(22) PCT Filed: Jan. 23, 2006
(86) PCT No.: PCT/GB06/00225

§ 371 Date: Aug. 10, 2007

(30) Foreign Application Priority Data
Feb. 11, 2005 (GB) 0502832.9

(51) Int. Cl. G06F 19/00; G01N 33/48

(52) U.S. Cl. 702/20

(57) ABSTRACT

A system for producing a display of the DNA identity of an individual comprises means for inputting a plurality of numbers and/or letters obtained through an individual's DNA analysis which are representative of a plurality of DNA loci; means for correlating inputted numbers and/or letters with predetermined colours; and means for producing a display with an array of coloured regions representative of each DNA locus and positioning said coloured regions in the same location of the display for each individual for which a display is produced.

### FIELD OF THE INVENTION

This invention relates to a system for producing a display of the DNA identity of an individual.

### BACKGROUND TO THE INVENTION

A number of test kits are available which, from an individual's biological material, can produce a series of numbers and/or letters corresponding to a number (typically 15 regions) of DNA loci. Many of these kits produce the result of identifying two numbers at each loci. The number of loci measured by each kit is chosen to be sufficient to precisely identify a given individual.

These test kits are often used in paternity tests. At each loci every person has two numbers, only one of which is passed on to his/her child. It therefore follows that a set of parents with numbers a, b and c, d respectively at a specific loci, can have children with the numbers a, c; a, d; d, c or b, d. No other numbers are permitted so that in this example, if the child has a number a, e at the same loci, then he/she is not the child of the man/woman with c, d. The numbers identified through such DNA tests are typically stored on databases. These databases may allow individual operators to either manually compare the numbers of an individual in the database with the numbers obtained from a recent test or may employ a search tool to identify numerical matches. This matching process may be complex to an unskilled member of the public whilst the resulting series of numbers for each individual may be difficult to understand and therefore of little practical everyday use.

### SUMMARY OF THE INVENTION

In a first broad independent aspect, the invention provides a system for producing a display of the DNA identity of an individual comprising means for inputting a plurality of numbers and/or letters obtained through an individual's DNA analysis which are representative of a plurality of DNA loci; means for correlating inputted numbers and/or letters with predetermined colours; and means for producing a display with an array of coloured regions representative of each DNA locus and positioning said coloured regions in the same location of the display for each individual for which a display is produced.

This system will have the advantage of producing visually readily comparable results for each individual. Individuals will be able to identify colour similarities and differences without requiring specialised training as would be the case for the prior art number print outs obtained from test kits directly. Another technical effect of the broad independent aspect is that the results may be better suited for scanning so that, for example, the results may be read in a similar manner as a barcode.

In a subsidiary aspect in accordance with the invention's broadest independent aspect, the coloured regions corresponding to a DNA locus are formed as a ring. This subsidiary feature will have the additional benefit of improving the manner in which the display may be read by a machine of a similar kind to a barcode scanner. It may also allow the display to be more readily scanned, even at angles where traditional barcodes could not be read properly.

In a further subsidiary aspect, the array of coloured regions is an array of concentric rings of different individual diameters which are spaced by non-coloured portions. This configuration will further improve the scanability of the display.

In a further subsidiary aspect, the display is produced on an article of clothing. This marks a complete departure from conventional thinking where barcodes are typically produced on flat cards. This will allow the presentation of the identification in a practical yet scanable form.

In a further subsidiary aspect, the system comprises a scanner to compare two or more individual displays. A colour sensitive optical scanner may preferably be used in order to obtain accurate comparisons. Such scanner would allow straightforward comparisons to be achieved without detailed DNA technology knowledge being required by individuals. Such comparisons might be straightforwardly implemented as airport security checks.

In a further subsidiary aspect, means are employed to identify similarities between two or more individual displays and highlight these similarities. This optional feature would allow related individuals to compare their similarities in detail

In a second broad independent aspect, the invention provides a method for assessing DNA identity of an individual comprising the steps of:
inputting to processing means a plurality of numbers and/or letters obtained through an individual's DNA analysis which are representative of a plurality of DNA loci;
correlating inputted numbers and/or letters with predetermined colours;
producing a display with an array of coloured regions representative of each DNA locus;
positioning said coloured regions in the same location of the display for each individual for which a display is produced;
scanning two or more individual displays; and
comparing them.

This would allow rapid genealogical comparisons to be carried out between individuals without requiring individuals to understand the complexities of DNA technology. This might be straightforwardly included into an airport security check.

### BRIEF DESCRIPTION OF THE FIGURES

FIG. 1 shows a display in the form of a chart produced by the system in accordance with a first embodiment. The chart is of a father.

FIG. 2 shows the chart of a mother in accordance with the first embodiment.

FIG. 3 shows a chart of a son of the mother and father of FIGS. 1 and 2 in accordance with a first embodiment of the invention.

### DETAILED DESCRIPTION OF THE FIGURES

The system used for producing a display of the DNA identity of an individual may employ a computer/user interface such as a keyboard in order to allow a user to input numbers and/or letters obtained through an individual's DNA analysis. The methods of obtaining these numbers and/or letters are well known in that particular art and needn't therefore be described in detail for the purposes of this invention. The computer may be set up with a look-up table which will allow the correlation between the inputted numbers and/or letters and a matrix of predetermined colours. This will allow consistent colours to be generated for every given number and/or letter. Once all the numbers and/or letters have been entered for a particular user and these numbers have been correlated to an individual's set of colours; the processing means may be set up to display an array of coloured regions representative of each DNA locus in a predetermined form.

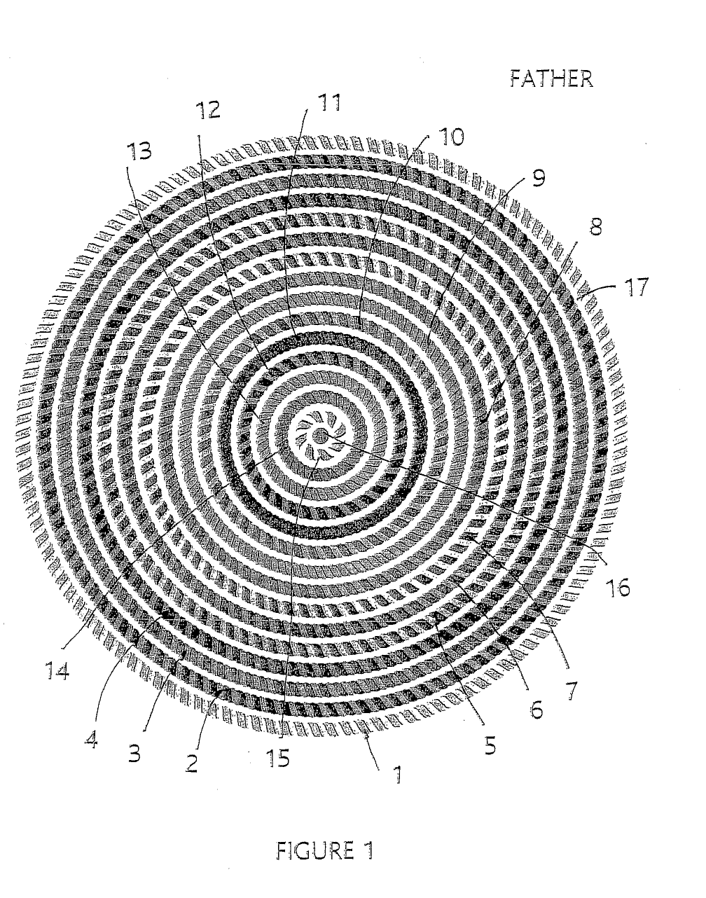

One predetermined form of display is illustrated in FIGS. 1, 2 and 3 for a father, mother and son respectively.

FIG. 1 shows an outer most ring 1 formed of a series of regions of alternate colours.

Ring 1 has alternate blue and yellow colours.

Ring 2 has alternate light blue and pink regions.

Ring 3 has alternate light blue and red regions.

Ring 4 has alternate green and pink regions.

Ring 5 has alternate blue and light pink regions.

Ring 6 has alternate light blue and blue regions.

Ring 7 has alternate blue and yellow regions.

Ring 8 has alternate blue and light blue regions.

Ring 9 has alternate light green and light blue regions.

Ring 10 has alternate blue and light blue regions.

Ring 11 has alternate blue and dark green regions.

Ring 12 has alternate pink and light blue regions.

Ring 13 has alternate light blue and light green regions.

Ring 14 has alternate green and light pink regions.

Ring 15 has alternate light green and yellow regions.

A blue dot 16 is located within ring 15.

Between each ring a space is provided such as that referenced 17 which is of sufficient length to clearly separate the individual rings for scanning purposes.

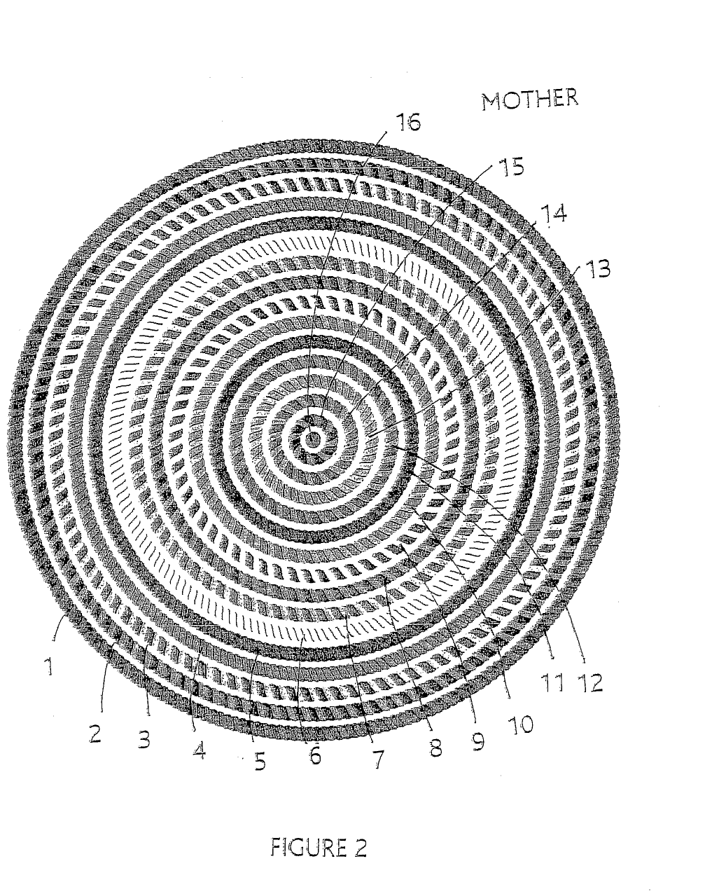

For ease of reference and comparison, rings of similar position between FIG. 2 and FIG. 1 have retained identical numerical references.

For the mother's chart of FIG. 2:

Ring 1 has alternate pink and red regions.

Ring 2 has a combination of pink and light blue regions.

Ring 3 has a combination of light blue and yellow regions.

Ring 4 has a combination of green and green regions.

Ring 5 has a combination of green and blue regions.

Ring 6 has a combination of yellow and yellow regions.

Ring 7 has a combination of yellow and red regions.

Ring 8 has a combination of light blue and pink regions.

Ring 9 has a combination of pink and yellow regions.

Ring 10 has a combination of light blue and green regions.

Ring 11 has a combination of light pink and blue regions.

Ring 12 has a combination of light green and blue regions.

Ring 13 has a combination of light green and green regions.

Ring 14 has a combination of grey and light pink regions.

Ring 15 has a combination of light blue and pink regions.

Dot 16 is red.

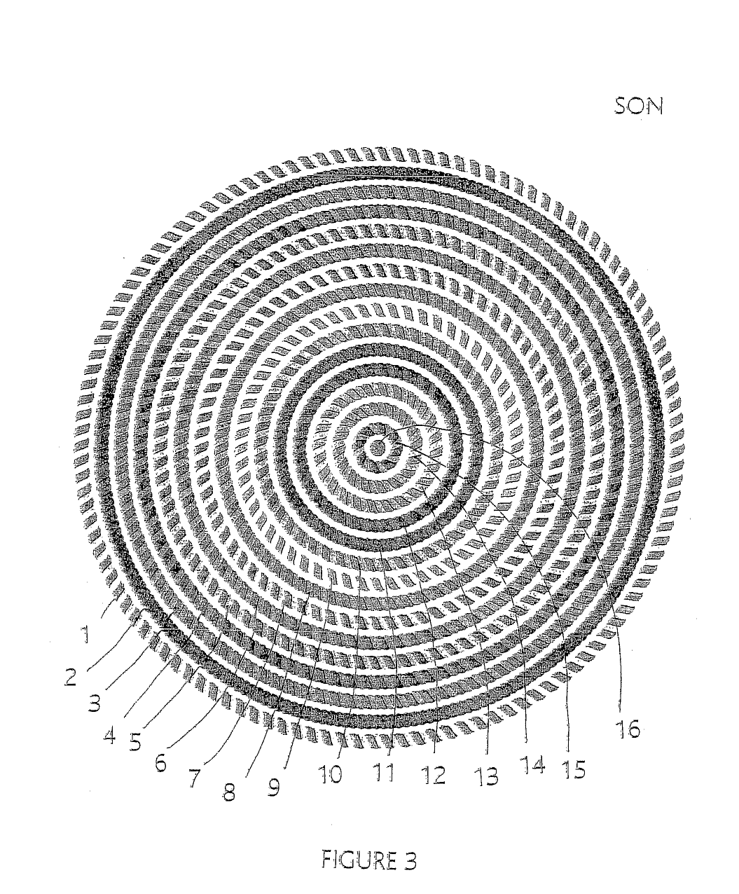

FIG. 3 shows the chart of a son of the father of FIG. 1 and the mother of FIG. 2.

Ring 1 is yellow and red.

Ring 2 is pink and pink.

Ring 3 is light blue and red.

Ring 4 is pink and green.

Ring 5 is blue and light pink.

Ring 6 is light blue and blue.

Ring 7 is blue and yellow.

Ring 8 is blue and light blue.

Ring 9 is yellow and light blue.

Ring 10 is blue and light blue.

Ring 11 is blue and dark green.

Ring 12 is pink and blue.

Ring 13 is light blue and light green.

Ring 14 is green and grey.

Ring 15 is light green and pink.

Dot 16 is blue.

Comparing the colour charts of FIGS. 1, 2 and 3 it is apparent that the son is the biological son of both parents. Ring 1 of the son's chart is formed of yellow and red having obtained the red from his mother and the yellow from his father. With regard to ring 2, the son will have obtained a pink region from each one of his parents. With regard to ring 3, he will have obtained a blue region from his mother and a red region from his father. With regard to ring 4, he will have obtained a pink region from his father and a green region from his mother. With regard to ring 5, he will have obtained a blue region from his mother and a yellow region from his father.

With regard to ring 6, he has the identical colour combination as his father which may be a mutation since his mother has a double yellow ring 6. With regard to ring 7, he has obtained a yellow from his mother and a blue from his father. With regard to ring 8, he has obtained a light blue from his mother and a blue from his father. With regard to ring 9, he has obtained a yellow from his mother and a blue from his father. With regard to ring 10, he has obtained a light blue from his mother and a blue from his father. With regard to ring 11, he has obtained a blue from his mother and a green from his father. With regard to ring 12, he has obtained a blue from his mother and a pink from his father. With regard to ring 13, he has obtained a light green from his mother and a light blue from his father. With regard to ring 14, he has obtained a grey from his mother and a pink from his father. With regard to ring 15, he has obtained a pink from his mother and a light green from his father.

The blue colour in spot 16 signifies that he is male and the red spot at the centre of the rings of his mother's chart symbolises that she is female.

Whilst the results of the system are shown in this embodiment as being a series of concentric rings which are spaced by non-coloured portions, the displays may be in the form of a histogram with two colour regions only for each bar drawn. It is also envisaged that these displays are printed onto a t-shirt or other article of clothing. It may also be incorporated in a tag. The invention also envisages that the colours are applied to 3 dimensional objects such as a cone, ball, a tube or any other appropriate object. The displays may also take the form of a bracelet or other jewellery as long as the order and colours are consistent for accurate scanning. The display may form part of an identity card as well.

1-9. (canceled)

10. A system for producing a display of the DNA identity of an individual comprising an input processor for inputting a plurality of numbers and/or letters obtained through an individuals DNA analysis which are representative of a plurality of DNA loci;
a correlator for correlating inputted numbers and/or letters with predetermined colors; and
a producer for producing a display with an array of colored regions representative of each DNA locus and positioning said colored regions in the same location of the display for each individual for which a display is produced.

11. A system according to claim 10, wherein the colored regions corresponding to a DNA locus are formed as a ring.

12. A system according to claim 11, wherein the array of colored regions is an array of concentric rings of different individual diameters which are spaced by non-colored portions.

13. A system according to claim 10, wherein the display is produced on an article of clothing.

14. A system according to claim 10, comprising a scanner for comparing at least two individual displays.

15. A system according to claim 14, wherein a comparator for identifying similarities between two or more individual displays and highlight these similarities.

16. A method for assessing DNA identity of an individual comprising the steps of:
inputting to a processor a plurality of numbers and/or letters obtained through an individuals DNA analysis which are representative of a plurality of DNA loci;
correlating inputted numbers and/or letters with predetermined colors;
producing a display with an array of colored regions representative of each DNA locus;
positioning said colored regions in the same location of the display for each individual for which a display is produced;
scanning two or more individual displays; and
comparing them.
